(19) United States
(12) Patent Application Publication
(10) Pub. No.: US 2015/0366478 A1
(43) Pub. Date: Dec. 24, 2015

(54) A METHOD AND SYSTEM FOR VECTOR ANALYSIS OF ELECTROCARDIOGRAM IN ASSESSMENT OF RISK OF SUDDEN CARDIAC DEATH (SCD) DUE TO ARRHYTHMOGENIC RIGHT VENTRICULAR DYSPLASIA/CARDIOMYOPATHY BY QUANTIFYING MICRO SCARS (I.E. "BITES") IN THREE DIMENSIONAL VECTOR LOOPS

(71) Applicant: Ivana I. VRANIC, Belgrade (RS)

(72) Inventor: Ivana I. VRANIC, Belgrade (RS)

(21) Appl. No.: 14/766,707

(22) PCT Filed: Apr. 5, 2013
(86) PCT No.: PCT/RS2013/000005

§ 371 Date: Aug. 7, 2015

(30) Foreign Application Priority Data
Feb. 8, 2013 (RS) P-2013/0048
Mar. 12, 2013 (RS) P-2013/0082

(51) Int. Cl. A61B 5/04; A61B 5/0472; A61B 5/0456

(52) U.S. Cl. CPC A61B 5/04011; A61B 5/04012; A61B 5/0456; A61B 5/0472

(57) ABSTRACT

Subject matter of the invention are a method and a system to provide diagnosis of arrhythmogenic right ventricular dysplasia—ARVC/D (and similar inherited conditions) that cause sudden cardiac death in otherwise healthy population. Innovative approach in this analysis is possibility to detect so called frusta forms or clinically silent forms. After recording on any ECG standard device, vector analysis is manipulated in strictly defined fashion. The shape and size of QRS complex seen in arrhythmogenic right ventricular dysplasia/cardiomyopathy is determined by the direction and magnitude of the spatially oriented electrical forces that have one common beginning those concerning vector bites. Besides one pathognomonic place there is a triangle of dysplasia to look for. The subject matter of the invention greatly increases the sensitivity and specificity of standard ECG device for early detection with a high likelihood ratio of certainty for a positive diagnosis.

### TECHNICAL FIELD

Subjected Invention belongs to the field of measurement for diagnostic purposes, especially for revealing, calculating or recording pulse rhythm or heart rhythm, as well as to the field of devices or method for data processing, especially adjusted for specific use. According to the International Patents Classification, the subject invention is labeled with the following classification symbols: A61J 5/00, A61J 5/024 and G06F 19/00/.

### BACKGROUND ART

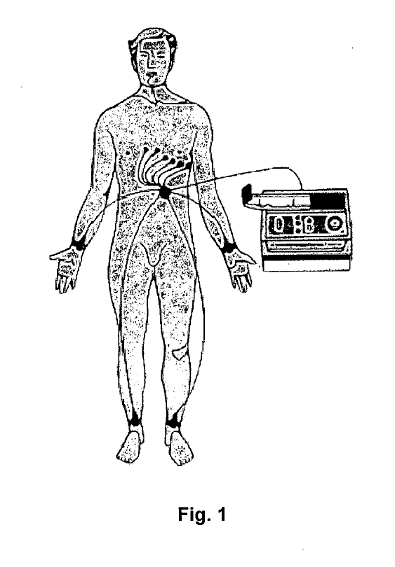

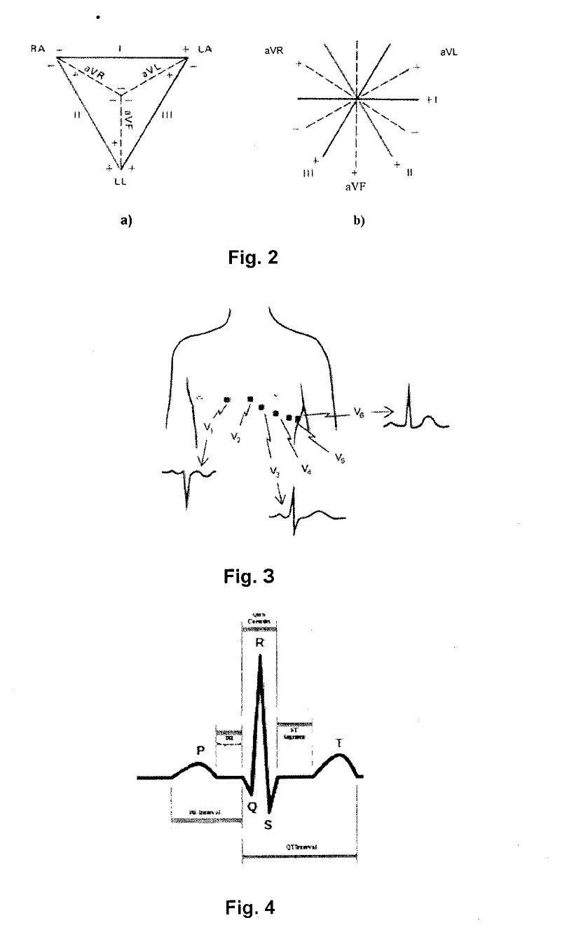

In the evaluation of real merit of heart signals expressed through vectors in time and space, a significant role belongs to electrode positioning on arbitrary agreed precise locations (FIG. 1). Depending on the type of ECG recorder, Einthoven bipolar leads D1, D2, and D3 with Baily triaxial system of amplification are in use: aVR, aVL, and aVF (which essentially do not change merits in mathematical sense), so obtained ECG inscription is being read from different angle perspective in total 6 extremity leads of 60 degrees angle (FIG. 2). Additional 6 leads are obtained from unipolar precordial system for measuring and these are following: V1, V2, V3, V4, V5 and V6 (FIG. 3).

ECG of a healthy person comprises the P wave, QRS complex (this complex comprises Q, R and S wave) and T wave (FIG. 4). Therefore, real waves are the P wave and the T wave. They can be positive, negative or biphasic. A Q wave can only be negative (there is no positive Q), then R wave can only be positive (there is no negative R) and the S wave can only be negative (there is no positive S). Specific wave type is determined on the basis of the R wave location (which is always positive) in the following manner: if a negative wave precedes the positive R wave then it is the Q wave. If a negative wave is followed after R wave then it is an S wave. Otherwise in normal QRS complex there is no more than one positive wave (and that is the R wave). If there are other R waves, they are mainly of pathological states and are marked R′. If other R waves exist, as it is generally in pathological states, they are denoted R′. If there is only one negative wave, without the positive R wave, then it is not clear whether it is the S or the Q wave, so it is called a QS wave or complex.

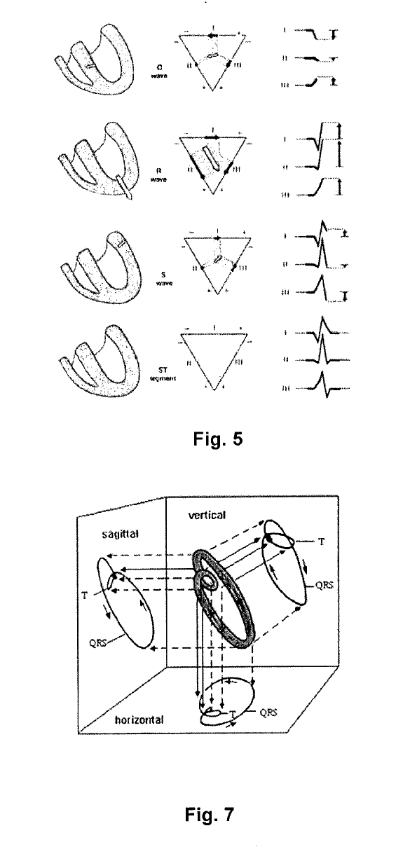

Fast and big changes in the size and direction of total dipole that are generated during ventricular depolarization result in QRS complex seen on ECG. Normal process is shown on FIG. 5. The start of ventricular depolarization usually happens on the left side of the medial part of interventricular septum. Analysis of heart dipole which is further generated by initial depolarization, with an aid of Einthoven's triangle, shows that this dipole has negative a component on lead I, a small negative component on lead II and a positive component on lead III. On FIG. 5, one may spot how dipoles make opposite deflection in individual leads. In such a way for example, the Q wave appears in leads I and II, but not in lead III. The second row in this picture shows both ventricles during instantaneous ventricular depolarization, in the moment of the largest number of total dipole with the most similar orientation. This phase creates large total dipole, which is responsible for the R wave on ECG. Such a dipole is almost parallel with lead II.

As it is shown, such dipole creates the big positive R wave on all three leads. The third line on FIG. 5, shows situation at the end of depolarizing spread through chambers and shows how small total dipole is at that very instant in the S wave creation. The S wave is not necessarily present in all leads. The bottom line shows that during ST segment formation, all cells within both chambers are in depolarized state. There is no wave of electrical activity that is transmitted through the heart tissue. There is no total dipole (i.e. difference between two body surface points regarding electrical potential). ECG record is flat at that point, i.e. isoelectrical).

Arrhythmogenic right ventricular dysplasia/cardiomyopathy (ARVD/C) is a genetic disorder which is associated with the concealed involvement of right chamber (RV) and its structural and functional changes (which are the result of such replacement of heart tissue with fat and fibrous tissue) and the electrical instability that causes ventricular arrhythmias and sudden cardiac death (SCD). Sudden cardiac death is a natural death caused by heart reasons preceded by loss of consciousness, which lasts about an hour and is a consequence of the acute heart symptoms. Sudden cardiac death often occurs in people who are generally healthy. Those who die a sudden death, probably are never aware of the potential risk they carry. Frequency of sudden cardiac death in unknown cause is estimated on 1/2000 people to 7/1000 people and no one is spared no matter of age, sex, geographic or socioeconomic position it is, somewhat less present in USA than in Europe. In Europe it is somewhat more frequent in the Mediterranean region, than in the north part of Europe, due to population migrations through history. Endemic regions are Veneto in northern Italy with 80% of incidence and island Naxos in Greece with 50% of incidence.

However, all noninvasive and invasive methods in consideration of the shape and function of RV have inherent limitations, due to the complex construction of its shape. The examination of RV could be extremely difficult task for a clinical doctor regarding its geometric complexity and the fact that it could be divided into three parts: inflow, outflow and RV body, which is falciform and abbreviated. Free wall of RV has more or less trabeculations, which in combination to its retrosternal position limit its precise chamber measuring and wall thickness. Tricuspid anterio-postrior systolic excursion (TAPSE) has been shown to correlate well with global function (in adult population) as EF LV whereas objectively assessed by radionuclide ventriculography (done by standard way). Recognizing mild, frusta, or localized forms, remains a clinical challenge. It is difficult to diagnose ARVC/D in a patient with minimal involvement of RV on heart ultrasound or contrast angiography. So far, only “V sign” on heart ultrasound, by author Dr Ivana I Vranic, has been attributed like pathognomonic. The recommended criteria for ARVC/D early phase detection from World Health Organization (and its working group on ARVC/D) have been found insufficient for this matter. The early identification of sport players who carry gen for ARVC/D plays a central role in the prevention of SCD during sports activities. Most frequent clinical manifestations of the disease are depolarize-repolarize changes on ECG, mainly localized in right precordial leads, global or regional morphologic or functional changes of RV and arrhythmias coming from RV. One should think of such disease even in asymptomatic person on the ground of positive ECG change and ventricular arrhythmia.

The individual diagnostics for sudden cardiac dying syndrome and assessing its risk in timely way remains an important and complex challenge.

It is well known in the art that electrocardiogram inscription so called ECG record, has its technical restrictions in diagnostic span, when it comes to the analysis of aggregate value of the vector trajectories in each instant of propagation of the electrical heart dipoles.

Conversely, VCG record is an attempt for objectification of relativity of acquisited difference in potential in all standard ECG devices (which have its technical restrictions) with an idea to maximize its diagnostic capacity. In simple words, vectorcardiogram represents a “tridimensional electrocardiogram”.

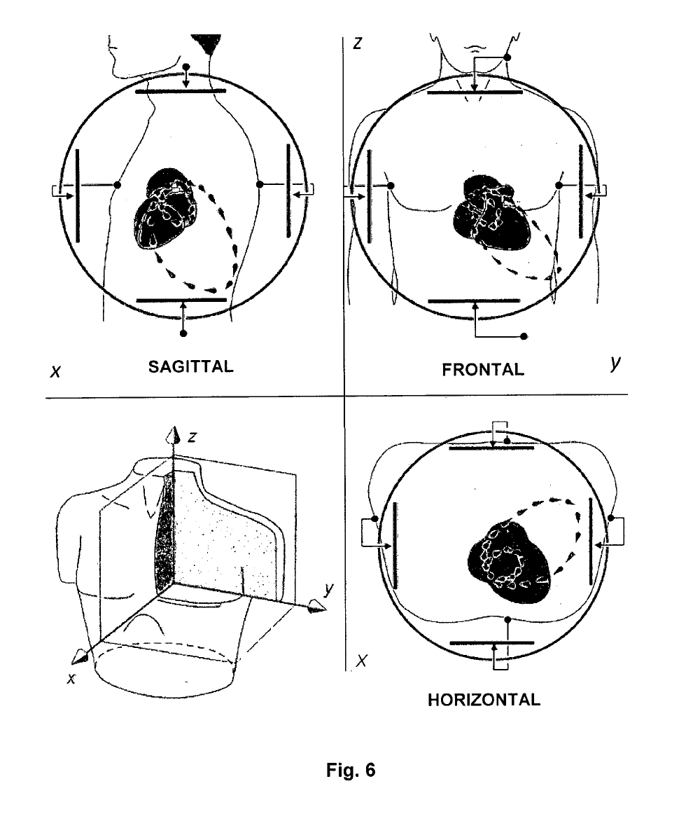

The vectorcardiographic appearance of ECG i.e. VCG record, represents sort of stereometric loop (closed curve or trajectory), which is usually shown in separate planes defined by appropriate axis (frontal: X,Y), (sagital: Y,Z) and (horizontal: X,Z) (see FIG. 6).

However, big input of significant information about signal value is not seen on the aspect from separate planes, but it is necessary to observe the loop in space. This offers the maximum usability of an analyzed signal in diagnostic and therapeutic sense, because vectorcardiography enables to overcome imperfection of classical ECG approach and provides a view of a larger real picture (see FIG. 7).

The reason for this is that every moment some part of atrial or ventricular heart muscle produces a small amount of the electrical force, directed up or down, right or left; and knowing that heart is a tridimensional structure, it is obvious that the electrical force also moves forwards or backwards. Spatially oriented electrical forces which are generated by heart per se appear in certain order, but not simultaneously.

The form and magnitude of the P wave, QRS complex, ST segment and T wave are set by management direction of aggregate vector and separate vector resultants determined by the location of unipolar precordial leads. Central direction of depolarization process reflects the sum of all vectors in each part of ventricular myocardium.

By searching all the available documentation, further documents were found from the field of measuring for diagnostic purposes: RS 49751 B, that is related to the device and method for wireless recording telecommunicating transmission of three special ECG leads and their processing, then YU 44792 B, that is related to ECG device with computer support which represents technical solution in 12 channel acquisition of ECG signal with digital output interface and YU 2217/86 A, that is related to ECG device for recording and transmission of all 12 leads on long distance by the use of phone or radio communication, and/or tape recording with reproduction; however, neither of these solutions is about VCG plotting, nor is it intended for assessment of risk of sudden cardiac death (SCD).

### Technical Problem

Accordingly, the present invention aims at addressing problem by providing solution with substantiation of the procedure and system which would enable diagnostic of sudden cardiac death syndrome (SCD) due to arrhythmogenic right ventricular dysplasia/cardiomyopathy for assessing its risk in timely manner in an individual.

### DISCLOSURE OF INVENTION

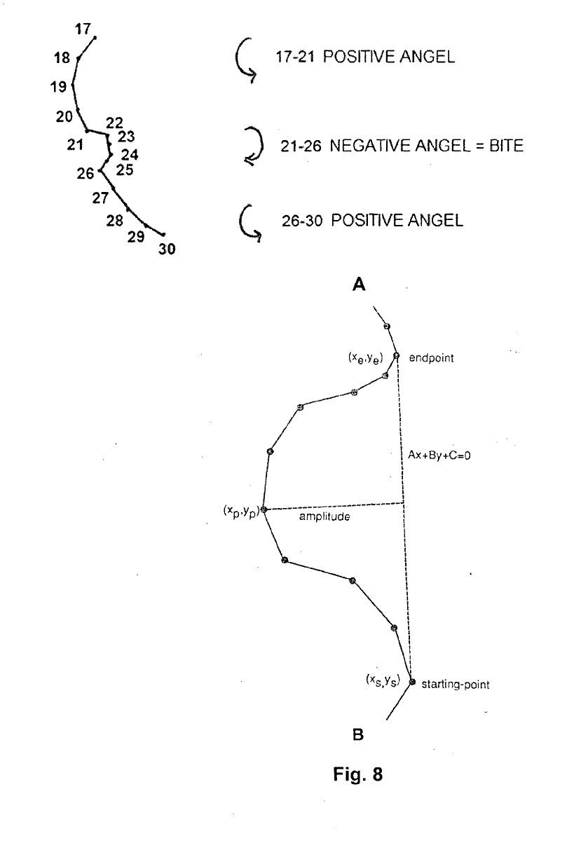

The above defined problem is solved using the procedure for vector analysis of electrocardiogram for the assessment of risk of sudden cardiac death syndrome (SCD) due to arrhythmogenic right ventricular dysplasia/cardiomyopathy by quantifying micro scars (i.e. “bites”), whereas each micro scar represents deviation from ideal curve as observed in three dimensional vector loop. Micro scars, or “bites” were first perceived in patients with diabetes mellitus, in whom small areas of fibrosis or necrosis in heart were found at autopsy, which was the basis for hypothesis that micro scars represent the expression of small myocardial lesion (see Edenbrandt L. et al. in Vectorcardiographic Bites, Journal of electrocardiology, Vol. 22, Oct. 4, 1989, page 325-331). In the aforementioned work it is specified that “bite” is considered present when a sector of vector loop changes its direction in contra route in comparison to the rest of major loop (for example, sector that rotates clockwise, unlike the rest of loop that rotates counterclockwise). The aforementioned sector is divided by several points. It is considered that each triplet of dotted row defines an angle, which is used as a checkup whether some sector of a loop rotates counterclockwise or not, taking into consideration that angle retains positive value if it rotates counterclockwise and takes negative value if it rotates clockwise (FIG. 8A). By further analysis of the observed sector loop and by the use of several iterations it is fortified with probability of 95% that the segment loop between points 21 and 26 exists a bite. Each bite characterizes amplitude which is defined as the longest distance from start and end point (see FIG. 8B) with corresponding duration and area. In the above mentioned manuscript it is indicated that the cut off for normal limits for bite amplitude are 0.12 mV (horizontal plane) and 0.14 mV (sagital plane), for duration 22 msec and (horizontal and sagital plane) and for the area 15% (horizontal) and 23% (sagital).

As it is cited, the present invention is concerned with the procedure of processing the vector analysis of electrocardiogram that achieves getting results in assessment of risk for sudden cardiac death. This is accomplished by postprocesual processing of recorded signal that is done after the acquisition of electrical heart signal on standard ECG recorder. The subject of analysis represents QRS loop, whose shape is changed in very specific and characteristic way in the early non manifest phase of disease in persons who are prone to sudden cardiac death inherited by birth.

The subject procedure can be used separately only after signal acquisition from ECG device, or alternatively inside such ECG device, in form of an upgrade in any such apparatus that is available on the world market. As an innovative solution, the subject invention represents a specific procedure of QRS loop analysis. Its inventiveness is comprised in solving the set of technical problems by means of automatic analysis i.e. pattern recognition shape of translatory trajectory of aggregate vector in time and space that occurs in arrhythmogenic right ventricular dysplasia/cardiomyopathy.

This procedure allows timely recognition of persons with the existence of risk of sudden cardiac death within otherwise healthy population—most frequently professional sportsmen and those whose phenotype gene expression is conditioned by the environment and life or work circumstances. The importance of the subject invention is reflected also in the fact that gene mutation (responsible for sudden cardiac death) has no influence on described analysis. Also, it is of vital importance that non manifest phase of the disease is only detectable by this analysis, in otherwise healthy person.

According to the invention, the procedure for vector analysis of electrocardiogram in assessing ground of existence for sudden cardiac death (SCD) due to arrhythmogenic right ventricular dysplasia/cardiomyopathy by quantifying micro scars i.e. “bites” in three dimensional vector loops, includes the following phases:
a) the first phase in which data about the electrical heart activity is collected from the electrocardiogram recording (ECG),
b) the second phase in which on the basis of assembled data vectorcardiogram (VCG) is generated,
c) the third phase in which the analysis of acquisited VCG is done, for the sake of assessing the existence of risk for SCD by quantifying micro scars i.e. “bites” in three dimensional vector loops,
d) the fourth phase in which the diagnosis ends and the finding is brought out whether the risk for SCD is established or not, and the subject finding is optionally, together with personal and other diagnostic data about the patient, stored in form of a database for later use,
   where each phase is comprised of one or more steps necessary for executing the activity defined in the above mentioned procedures' phases according to the invention.

In one embodiment of the present invention, the present invention at its first phase, takes data collection online/offline about the electrical heart activity recorded by electrocardiogram (ECG).

In another embodiment of the present invention, the part of invention for data collection of the electrical activity of heart could be done online by direct loading from apparatus or remote loading, while offline data collection of the electrical activity of heart could be done by scanning or loading stored data.

In another embodiment of the present invention for data collection of the electrical activity of heart which is done by scanning, picture selection and manual or automatic search on finding ECG leads and settings of the procured leads are made.

In another embodiment of the present invention, instead of creating vectorcardiogram (VCG) on the basis of ECG recorded and collected data of the electrical activity of heart, those data are obtained by vectorcardiography in the third phase.

Accordingly, the method for vector analysis of electrocardiogram in assessment of the risk of sudden cardiac death (SCD) due to arrhythmogenic right ventricular dysplasia/cardiomyopathy by quantifying micro scars (i.e. “bites”) in three dimensional vector loops, according to the invention is comprised of the following steps:

choice of way data procurement is made, which may be online/offline,
online data acquisition, which can be direct loading or remote loading,
direct loading of data immediately accessed from particular ECG device,
remote loading that is achieved by some way of wireless connection,
offline downloading data, obtained by ECG recordings scanning or by loading stored data,
scanning of ECG tracings desirable in perpendicular way,
picture selection for scanning from digitalized signal finding or quality scan picture of ECG record in paper,
choice of search fashion for adequate ECG leads between manual or automatic options,
manual search for ECG leads that is accomplished by following the closest lead from the point of selection,
automatic search that allows finding three or more than three leads at once,
settings of acquired leads in which potential errors will be removed and key points determined: horizontal null, beginning of Q part of loop, beginning of R part of loop and beginning and end of S part of loop,
loading stored data by downloading appropriate archived database,
plotting of VCG, that follows the step of direct loading, remote loading, settings of acquired leads or downloading stored database, and is being done by application of inverse orthogonal projection that converts 2D VCG into 3D VCG (3D loop) and separate pieces of Q, R and S parts of loop are delineated,
choice whether transformations and settings as optional step are needed, if positive answer is achieved then access to executing desired transformations and settings is done, such as magnifying or decreasing the loop, moving it, rotating it and so forth,
auditing data consistency by which it is determined if there is a deformation in real data of acquired values; in the case of a positive answer they are abandoned for further analysis and an error is registered, by which the process terminates; in the case of a negative answer transfer to search for the obvious signs of disease is made,
search for the obvious signs of disease such as manifest phase and obvious phase of disease; in case these are found, then the risk of SCD is established, which terminates further analysis; in the case of a negative answer transfer to checking the S part of loop is made,
checking the S part of loop with searching S part for deviation from expected trajectory by which the loop should pass in such manner that compares its length with beforehand defined border appointed in database; if a deviation for beforehand defined percent higher than defined border, then the risk of SCD is established and the process terminates; if a deviation is found in defined epsilon environment of beforehand appointed border, then transfer to the R part of loop is made together with forwarding specific indication of the existence of the risk of SCD,
checking the R part of loop that is done on data obtained from axonometric transformation of loop projections on a plane with the best view of a bite, by looking for peaks r′R or Rr′ and/or abrupt change of itinerary of vector sector loop in increasing or decreasing R part, respectively; if an r′ peak is found then the risk of SCD is established, which terminates further analysis; if only the change of itinerary is found then it is checked whether the indication for potential risk of SCD was forwarded from checking S part of the loop; if the answer is positive then the risk of SCD is established, which terminates further analysis; if the answer is negative or if nothing was found, then no risk of SCD is established, which terminates further analysis.

Besides the above mentioned procedure, the subject of the invention represents the appropriate system for vector analysis of electrocardiogram in assessment of the risk of sudden cardiac death (SCD) due to arrhythmogenic right ventricular dysplasia/cardiomyopathy by quantifying micro scars (i.e. “bites”) in three dimensional vector loops. Mentioned system is comprised of the following units:
a) unit for data collection about the electrical activity of heart that is recorded on electrocardiogram (ECG),
b) unit in which vectorcardiogram (VCG) is generated on the basis of collected data,
c) unit in which the analysis of acquisited vectorcardiogram for the establishing the risk of SCD by quantifying micro scars (i.e. “bites”) in three dimensional vector loops,
d) unit in which the result of diagnostics related to estimated risk of SCD is handed out and is optionally together with patient data of personal and other nature stored in a database for later use.

In one preferred embodiment of the present invention, the system is integrated within the ECG device, as an adequate upgrade.

In another preferred embodiment of the present invention, the adequate unit represents personal computer.

The present invention is to be further thoroughly described on the basis of the attached draft below by which:

FIG. 1 shows the procedure of recording electrogardiogram by taking inscription of the electrical heart activity with standard ECG device, either with analog or digital signal output;

FIG. 2 shows triaxial system;

FIG. 3 shows order of precordial electrode system

FIG. 4 shows appearance of a heart cycle

FIG. 5 shows schematic representation of Einthoven's triangle;

FIG. 6 represents two dimensional display of vector loop in certain planes;

FIG. 7 represents three dimensional vector loop;

FIG. 8A represent vector loops with bite, and FIG. 8B denotes bite amplitude;

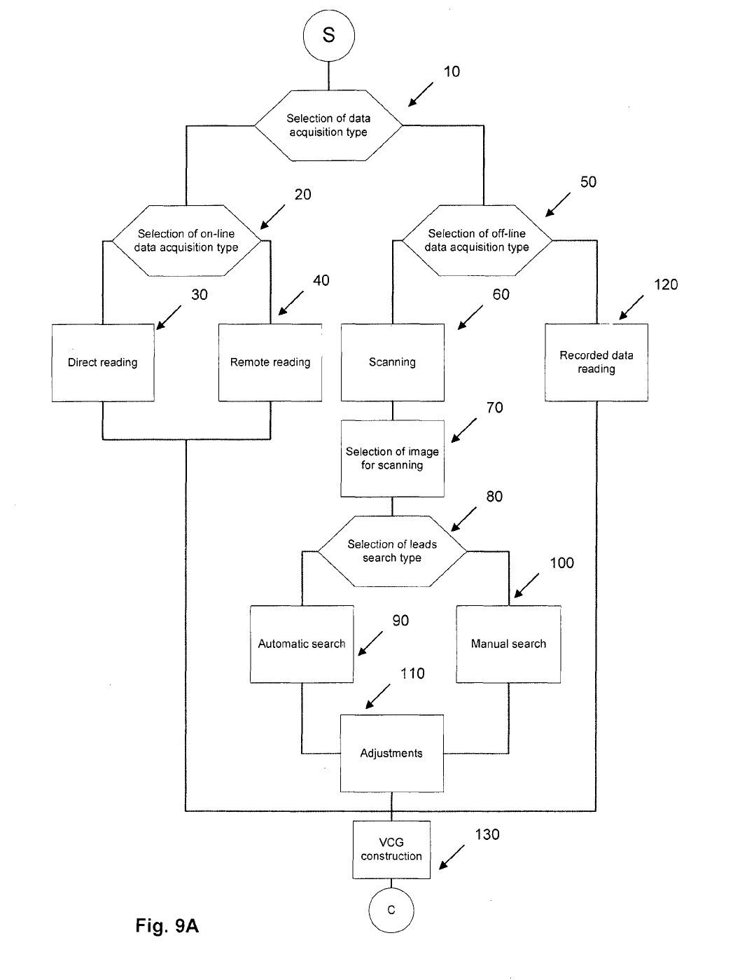

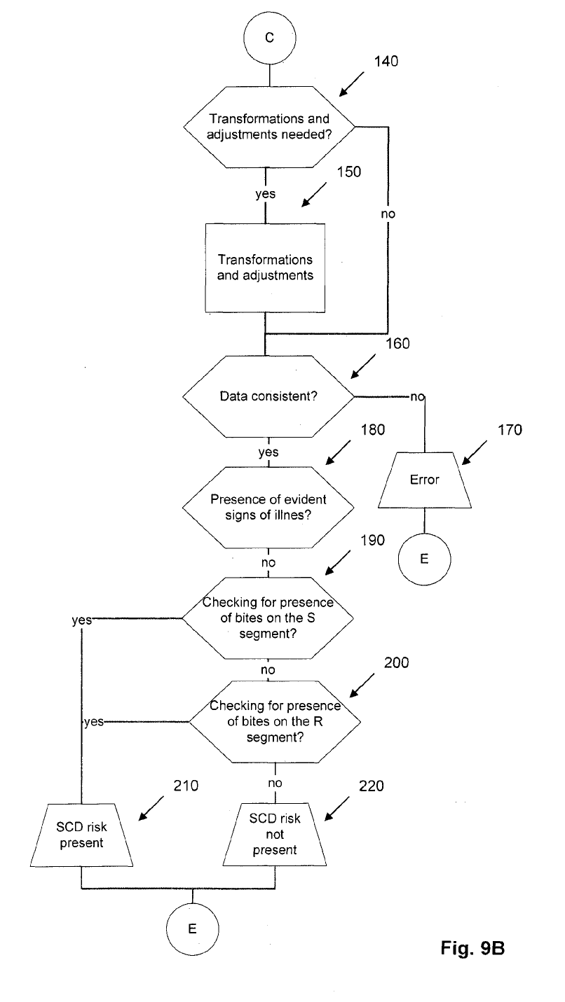

FIGS. 9A and 9B represent algorithmic chart of procedure according to invention;

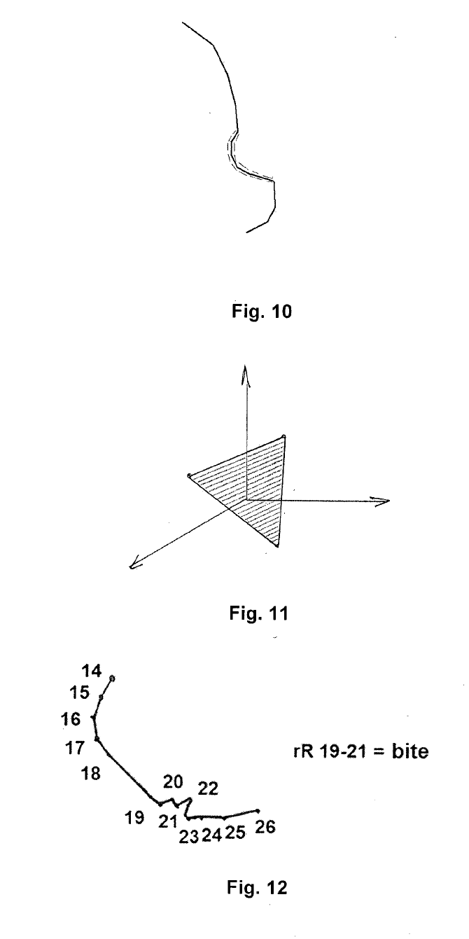

FIG. 10 represents deviation of expected itinerary;

FIG. 11 represents axonometric transformation of loop projections on a plane;

FIG. 12 represents peaks r′R or Rr′ in a heart cycle.

As mentioned above, subject invention refers to method for vector analysis of electrocardiogram in assessment of the risk of sudden cardiac death (SCD) due to arrhythmogenic right ventricular dysplasia/cardiomyopathy by quantifying micro scars (i.e. “bites”) in three dimensional vector loops according to the invention which achieves the recognition of arrhythmogenic right ventricular dysplasia/cardiomyopathy (and similar congenital states) that cause sudden cardiac death in otherwise healthy population. The subject invention will be further thoroughly described below in the first place from the reference of FIGS. 9A and 9B which show steps that constitute procedure according to the invention.

Accordingly, the procedure according to the invention includes the following steps:
choice of way of data downloading 10: in step 10 the choice of way of data downloading that is necessary for this procedure is particularized. The signal acquisition is a process of accessing the information needed for further diagnostics. ECG information could be accessed from several sources. Thus, for example, new finding can be acquired directly from apparatus, or from the picture of existing finding and also digitally stored finding may be used. In general, data downloading could be in real time, i.e. on line or, opposite to, previously obtained data may be used, i.e. off line acquiring can be performed while it is emphasized that further stream depends on elected way of loading.
on-line data downloading 20: if this way is selected of data downloading, next step of the procedure is related to selection between direct data downloading 30 or remote data downloading 40, whereas further stream depends on elected way of loading.
direct data downloading 30: if the way of data downloading is direct by from a device, then in this step it is possible by using different streams, necessary because of diversity of ECG devices, to access data directly from ECG apparatus. Streams (wire connections) are made by manufacturer companies or in agreement with them. In case of direct data downloading, ECG device is in direct, physical contact with a personal computer, which contains the appropriate software for executing the analysis procedure according to the invention. Physical connection is achieved by cables, by using more than one standard ways such as serial port, parallel port, USB and similar.
remote data downloading 40: if remote data downloading is chosen then it is generated through some way of wireless connection. As such, there are different standards for wireless connection with PC, such as WiFi, Bluetooth, infrared and similar.
on-line data downloading 50: if this way of data downloading is elected, the further step of the procedure would relate to selection between scanning of ECG finding 60 or downloading stored data 120, whereas further stream depends on chosen way of loading.
scanning 60: If as an off line way of data downloading is elected scanning, then it is performed by scanner which is an optical input device that allows raw data such as drawings, photo or text to be transferred in suitable form of digital informations. For the subject invention it is necessary to scan ECG finding in perpendicular manner (to avoid errors).
picture election for scanning 70: For scanning, it takes selection of the appropriate picture that allows ECG finding to be loaded, such as digitally generated finding or quality scan picture of the existing ECG finding on paper. ECG finding can be digitalized in any form of the usual picture formats. For acquisition, more than one picture can be used at once if scanning is done in several parts or for comparative purposes.
selection of way of search 80: After pictures election and scanning, it is necessary to find suitable ECG leads, therefore the next step is dedicated to choosing between manual search 90 or, alternatively, automatic search 100.
search of ECG leads (manual search) 90: if manual search is chosen, the start is made from the scanned picture, which at the beginning is a simple information on picture color and lightness of each part of it. By filtering from that bunch of information we gather what is necessary, and those are ECG leads. Channels are selected by user. Leads are found by following to the nearest lead of the selected point.
Automatic search 100: As an alternative to previous step, i.e. the second option represents automatic search, which means that automatization is used depending on user choice. Automatic search of leads is executed depending of selected lead. Considering that leads on ECG finding are organized by groups of three leads, it enables three or more leads to be found at the same time.
adjustments of acquired leads 110: Regardless of whether manual or automatic search is performed, after which leads are obtained, in this step the adjustments are possible, regarding the elimination of eventual errors and finding key points. First one is horizontal null, that is automatically adjusted, but can be manually fitted. Other key points are related to QRS complex. Those points are: starting point of Q part of loop, beginning of R part of loop and beginning and end of S part of loop. Those are automatically found, but it is recommended to set them manually.
downloading of stored data 120: if way of downloading stored data is selected it is necessary to download suitable archived database. The type of archived database depends on the type of acquisition. If acquisition was made from device then it is necessary to record finding first. If acquisition was made from a picture, that means that, for example, it was adjusted as explained in steps 60-100.
plotting of VCG 130: Independently of selected way of data acquisition, therefore regardless of whether this step is preceded by step 30, step 40, step 110 or step 120; it is this step in which transformation from ECG to VCG is done, by application of inverse orthogonal projection that converts 2D VCG into 3D VCG. Accordingly, on the basis of loaded data from leads of ECG vector view is generated, i.e. 3D loop. 3D loop is obtained from separate dimensions that are generated from ECG leads that are most alike orthogonal projection of vectors suited to specific dimension. After all generated vectors which define 3D loop, that loop is plotted by parts, that is separate plotting of Q, R and S parts of loop are inscribed.
choice whether transformations and adjustments are needed 140: This step is optional, and in a case of a positive answer, the access to desired transformations and adjustments 150 is made. Namely, after generating vectors for plotting the loop it is possible to transform the loop in several ways. Magnifying or decreasing the loop, up to the certain extent is possible. It is also possible to move the loop in the plane of view for the sake of easier magnifying of desired parts. Also, it is possible to rotate it for a full circle in all three dimensions. It is possible to choose what is plotted, i.e. which parts of Q, R and S and the rest of loop are inscribed. All transformations and change of adjustments has no influence on further diagnostics, but serve only for a better view of loop.
checking data for consistency 160: The analysis in this step begins with checking on data for consistency in ECG derived informations in different leads. If it is proved that there is a deformation of real merits in loaded data, then they are rejected for further analysis, because they are not valid, so error 170 is reported and by that procedure ends. In similar fashion checking on general superposition between QRS complexes from different leads.
search for the obvious signs of disease 180: If step 160 confirms that data are consistent, after such evaluation is completed, search for the obvious signs of disease is being made, such as manifest and obvious phase of disease. Here the isolated dilatation of right chamber is the case, which is presented by wall thinning and fibro-fatty infiltration of free heart wall with partial loss of contractility and generating zone of ballooning effect, so called “bulgings” in systole (that is in heart cycle phase in which heart contracts), and is proven by: a) heart ultrasolund (echocardiography examination), B) nuclear magnetic resonance of heart or C) by heart biopsy, more precisely of right heart. In case those signs are found, diagnostics ends and the result is positive, i.e. risk for SCD is recognized 210.
checking the S part of loop 190: If the previous step did not find any obvious signs of disease, then checking the S part of loop for deviation from expected trajectory is made in this step (see FIG. 10). Whether there is a deviation is concluded by comparing the length of S part of loop with in advance defined border set in a database. If a deviation for beforehand defined percent is higher than defined border, diagnostics terminates and the result is positive i.e. the risk of SCD is established 210. If a deviation is found in defined epsilon environment of beforehand appointed border, then transit to the R part of loop is made 200 altogether with specific indication for the existence of risk for SCD which is forwarded depending on the result of the step 200. The additional evaluation is executed to reject existence of the risk of SCD because deviation is slightly smaller than beforehand appointed border, or not to accept existence of the risk of SCD because deviation is slightly bigger from beforehand appointed border, i.e. not to pronounce persons with a risk of SCD healthy ones with no risk, and those with no risk of SCD pronounce potentially in danger.
checking the R part of loop 200: Contrary to previous step 190, when the checking of S part of loop is done on it, in step 200 checking the R part of loop is done on data obtained from axonometric transformation of loop (see FIG. 11) in such manner of finding axonometric transformation with best view on a bite. Seeking for peaks r′R or Rr′ (see FIG. 12) and/or abrupt change of itinerary of vector sector loop in increasing or decreasing R part, respectively. If an r′ peak is found, the result is positive and the risk of SCD is established 210. If the answer is negative or if nothing was found, the diagnostic procedure terminates, the result is negative and no risk of SCD is established 220.

Accordingly, two possible outcomes, according to the invention are:
the risk for SCD is established 210: If obtained results are positive, the confirmation of diagnosis is written for presence of SCD risk and on what basis the conclusion has been reached.
no risk for SCD is established 220: If obtained results are negative, then only written message is given.

Obtained results together with personal and other diagnostic data about the patient can be stored in a database for further use. That is the end of performing the procedure according to the invention.

The procedure according to the invention represents the basis of making suitable software which would provide the estimation of grounds for sudden cardiac death SCD due to arrhythmogenic right ventricular dysplasia/cardiomyopathy by quantifying micro scars (i.e. “bites”) in three dimensional vector loops in reliable and repeatable way. Accordingly, the subject invention in the first place has its use in the field of medicine, especially in medical diagnostics.

It is understandable that on the basis of the description of this invention different kinds of performing the procedure and system according to the invention can be realized, while remaining within the scope of the invention which is defined in the attached patent claims.

1. Method for vector analysis of electrocardiogram in the assessment of the risk of sudden cardiac death (SCD) due to arrhythmogenic right ventricular dysplasia/cardiomyopathy by quantifying micro scars (i.e. “bites”) in three dimensional vector loops, characterized in that it includes the following phases:
a) the first phase in which collecting information about the electrical activity of heart recorded on electrocardiogram (ECG) is performed by an unit for data collecting about the electrical activity of heart recorded on electrocardiogram (ECG);
b) the second phase in which based on the collected data vectorcardiogram (VCG) is generated by an unit for generating vectorcardiogram (VCG) on the basis of collected data;
c) the third phase in which the analysis of obtained vectorcardiogram due to arrhythmogenic right ventricular dysplasia/cardiomyopathy by quantifying micro scars (i.e. “bites”) in three dimensional vector loop, is performed by an unit for the analysis of obtained vectorcardiogram for the sake of estimating the existence of the risk of SCD by quantifying micro scars (i.e. “bites”) in three dimensional vector loop; and
d) the fourth phase in which diagnostics ends and the result on whether the risk of SCD is established or not is issued, and the acquired result is optionally, together with personal and other diagnostic data about the patient, stored in a database for further use is performed by an unit in which on the basis of such diagnosis results are printed and issued out, providing basis on which the risk of SCD is established or not, and the subject finding is optionally, together with personal and other diagnostic data about the patient, stored in form of database for later use.

2. Method according to claim 1, characterized in that it contains the following steps in the third phase contains the following steps:
search for the obvious signs of disease (180) such as manifest and obvious phase of disease; in case these signs are found, the risk of SCD is established (210);
checking the S part of loop (190) if in previous step (180) no obvious signs of disease were found, seeking for deviation from expected trajectory; if a deviation for beforehand defined percent is higher than defined border then the risk of SCD is established (210); if a deviation is found in defined epsilon environment of beforehand appointed border, then transfer to the R part of loop is made (200) together with specific indication for the existence of potential risk of SCD; and
checking the R part of loop (200) is done on data obtained from axonometric transformation of loop in such manner of finding axonometric transformation with best view on a bite by seeking for peaks r′R or Rr′ and/or abrupt change of itinerary of vector sector loop in increasing or decreasing R part, respectively; if an r′ peak is found, the risk of SCD is established (210); if only the change of loop itinerary is found, then it is checked if specific indication is forwarded from S part of loop for the existence of the risk of SCD (190); if the answer is positive then the risk of SCD is established (210); if the answer is negative or if nothing was found, no risk of SCD is established (220).

3. Method according to claim 1, characterized in that collecting data about the electrical activity of heart recorded on electrocardiogram (ECG) can be done on line or off line.

4. Method according to claim 3, characterized in that on line collecting of data about the electrical activity of heart which can be done by direct loading from apparatus or remote loading, where an off line collecting of data about the electrical activity of heart can be done by scanning or loading of stored data.

5. Method according to claim 4, characterized in that collecting data about the electrical activity of heart done by scanning needs the selection of a picture for scanning, after which manual or automatic search of ECG finding, specifically leads and setting up the leads, is performed.

6. Method according to claim 1, characterized in that instead of vectorcardiogram (VCG) obtained on the basis of collected data about the electrical activity of heart recorded by electrocardiogram (ECG), in third phase the data about the electrical activity of heart are acquired by vectorcardiography.

7. Method for vector analysis of electrocardiogram in assessment of the risk of sudden cardiac death (SCD) due to arrhythmogenic right ventricular dysplasia/cardiomyopathy by quantifying micro scars (i.e. “bites”) in three dimensional vector loops characterized in that it is performed by the system containing an unit for data collecting about the electrical activity of heart recorded on electrocardiogram (ECG), an unit in which on the basis of gathered data vectorcardiogram (VCG) is generated, an unit in which the analysis of obtained vectorcardiogram is performed for the sake of estimating the existence of the risk of SCD by quantifying micro scars (i.e. “bites”) in three dimensional vector loop and an unit in which on the basis of such diagnosis results are printed and issued out, providing basis on which the risk of SCD is established or not, and the subject finding is optionally, together with personal and other diagnostic data about the patient, stored in form of database for later use, wherein the said method contains the following steps:
choice of way of data loading (10), either online (20) or offline (50);
online data loading (20), which can be direct data loading (30) or remote data loading (40);
direct data loading (30) which is achieved by direct access loading from ECG device;
remote data loading (40) that is achieved by some form of wireless connection;
offline data loading (50), which can be done by scanning of ECG finding (60) or loading stored data (120);
scanning (60) of ECG finding desirably in perpendicular fashion;
selection of picture for scanning (70) from digitally generated finding or from quality scan picture of ECG finding in paper;
selection of way of search (80) for adequate ECG leads between manual (90) or automatic search (100);
manual search of ECG leads (90) which is done by following the closest lead from the point of selection;
automatic search (100) which enables to find three and more leads at the same time;
adjustments of the obtained lead (110) by which potential errors are removed and key point are found: horizontal null, beginning of Q part of loop, beginning of R part of loop and beginning and end of S part of loop;
loading stored data (120) which is done by loading suitable archived database;
plotting of VCG, that follows the steps (30), (40), (110) and (120) by application of inverse orthogonal projection that converts 2D VCG into 3D VCG (3D loop) and separate pieces of Q, R and S parts of loop are delineated;
choice if transformations and adjustments (140) are needed is optional step, if positive answer is achieved then access to executing desired transformations and settings is done, such as magnifying or decreasing the loop, moving it, rotating it and so forth;
auditing data consistency (160) by which it is determined if there is a deformation in real data of acquired values; in case of a positive answer, they are abandoned for further analysis and an error is registered (170), by which the process terminates; in case of a negative answer transfer to search for the obvious signs of disease is made (180);
search for the obvious signs of disease (180) such as manifest phase and obvious phase of disease; in case these are found, then the risk of SCD is established (210), which terminates further analysis; in case of a negative answer, transfer to checking the S part of loop is made (190);
checking the S part of loop (190) with searching S part for deviation from expected trajectory by which the loop should pass in such manner that compares its length with beforehand defined border appointed in database; if a deviation for beforehand defined percent higher than defined border, then the risk of SCD is established (210) and the process terminates; if a deviation is found in defined epsilon environment of beforehand appointed border, then transit to the R part of loop is made (200) together with forwarding specific indication of the existence of the risk of SCD; and
checking the R part of loop (200) that is done on data obtained from axonometric transformation of loop projections on a plane with the best view on a bite, by looking for peaks r′R or Rr′ and/or abrupt change of itinerary of vector sector loop in increasing or decreasing R part, respectively; if an r′ peak is found then the risk of SCD is established (210), which terminates further analysis; if only the change of itinerary is found then it is checked whether the indication for the potential risk of SCD was forwarded from checking S part of the loop (190); if the answer is positive, then the risk of SCD is established (210), which terminates further analysis; if the answer is negative or if nothing was found, then no risk of SCD is established (220), which terminates further analysis.

8. System for vector analysis of electrocardiogram in assessment of the risk of sudden cardiac death (SCD) due to arrhythmogenic right ventricular dysplasia/cardiomyopathy by quantifying micro scars (i.e. “bites”) in three dimensional vector loops, characterized in that it contains the following units:
a) unit for data collecting about the electrical activity of heart recorded on electrocardiogram (ECG)
b) unit in which on the basis of gathered data vectorcardiogram (VCG) is generated,
c) unit in which the analysis of obtained vectorcardiogram is performed for the sake of estimating the existence of the risk of SCD by quantifying micro scars (i.e. “bites”) in three dimensional vector loop,
d) unit in which on the basis of such diagnosis results are printed and issued out, providing basis on which the risk of SCD is established or not, and the subject finding is optionally, together with personal and other diagnostic data about the patient, stored in form of database for later use.

9. System according to claim 8, characterized in that it is being integrated in the particular ECG device, in the form of a suitable upgrade.

10. System according to claim 8, characterized in that at least one of corresponding units is personal computer.
